(19) United States
(12) Patent Application Publication
(10) Pub. No.: US 2007/0098661 A1
(43) Pub. Date: May 3, 2007

(54) Liquid to powder composition

(76) Inventor: Bartley Dean Maxon, St. Louis, MI (US)

Correspondence Address: Dow Corning Corporation;Mail Number C01232, 2200 W. Salzburg Road, P.O. Box 994, Midland, MI, 48686-0994, US

(21) Appl. No.: 11/521,245

(22) Filed: Sep. 14, 2006

Related U.S. Application Data
(60) Provisional application No. 60/733,290, filed on Nov. 3, 2005.

(51) Int. Cl. A61K 8/89

(52) U.S. Cl. 424/70.12

(57) ABSTRACT

The invention relates to silicone liquid-to-powder compositions for topical skincare applications. More specifically the invention relates to topical compositions comprising: a cosmetically acceptable powder, a silicone elastomer; a volatile fluid and a nonvolatile silicone fluid. The resulting product can be in the form of a viscous cream, lotion or putty which, when is applied to the skin, converts quickly to a dry powder.

### CROSS-REFERENCE TO RELATED APPLICATIONS

NONE

### BACKGROUND OF THE INVENTION

The invention relates to silicone liquid-to-powder compositions for topical skincare applications. More specifically the invention relates to topical compositions comprising: a cosmetically acceptable powder, a silicone elastomer; a volatile fluid and a nonvolatile silicone fluid. The resulting product can be in the form of a viscous cream, lotion or putty which, when is applied to the skin, converts quickly to a dry powder.

There are a number of skincare products that go on in fluid form, and eventually transform from liquid to powder on the skin, however these are based on a volatile carriers, usually an alcohol or volatile hydrocarbons. These products have disadvantages such as the volatiles can strip the skin; the formulas can leave a gritty feel on the skin and leave an unpleasant whitish residue upon drying; and because of the selected components, the products do not ordinarily have the smooth, creamy esthetics or skin feel desired, nor is their level of evaporation upon application so thorough as to leave a substantially completely dry product.

Because of this, there thus remains a need for a liquid-to-powder composition that provides all the desired characteristics of a luxurious fluid product and powder product, without the disadvantages that have typically been associated with such products.

### BRIEF SUMMARY OF THE INVENTION

This invention pertains to a liquid-to-powder composition comprising

(A) at least one cosmetically acceptable powder

(B) at least one crosslinked silicone elastomer;

(C) at least one volatile fluid and;

(D) at least one nonvolatile silicone fluid.

The compositions of the invention provide a suitable vehicle for infant or adult powder based skin care products, as well as for delivery of pharmaceutical actives. The resulting compositions have the elegant, smooth spreading properties of a cream or viscous putty, but which upon application to the skin convert to a dry, silky-feeling powder.

### DETAILED DESCRIPTION OF THE INVENTION

Component (A) in the liquid-to-powder formulation is a cosmetically acceptable powder. Examples of powders useful herein include starch, talc, silica, mica, sugars, inorganic pigments, and natural skin treatment powders such as oatmeal, soy powder and mixtures thereof. Preferred are those typically found in infant powder based skin care product such as cornstarch or talc. Typically the powders have a particle size of 5 to 600 microns, alternatively 80 to 300 microns and are commercially available. The cosmetically acceptable powder useful herein are typically those that conform to the specifications of the Cosmetic and Toiletry and Fragrance Association.

Starch powders useful herein include cornstarch, tapioca granules, wheat starch and potato starches. Corn starch are those carbohydrate polymers derived of corn of various types, typically composed of 25% amylose and 75% amylopectin. These starch granules will differ in size and shape, depending on the plant source. Corn starch granules are slightly larger (approximately 15 μm), and round to polygonal. Tapioca granules are even larger (approximately 20 μm), with rounded shapes that are truncated at one end. Wheat starch tends to cluster in several size ranges: Normal granules are approximately 18 μm; larger granules average about 24 μm; and smaller granules average approximately 7 to 8 μm, with round to elliptical shapes. Potato starches are oval and very large, averaging 30 to 50 μm. However through processing, these powders can be micronized to particle sizes of less than 10 μm.

The talc which is useful herein is typically a white, odorless, fine powder ground from a naturally occurring rock ore and typically consist mainly of hydrous magnesium with the remainder being naturally associated mineral such as calcite, chlorite, dolomite, kaolin and magnesite. Typically the particle size is such that 100% passes through a 60 mesh screen and not less than 99% passes through a 100 mesh screen and at least 98% passes through a 200 mesh screen.

Micas useful herein include, but are not limited to muscovite, phlogopite, tiotite, sericite, lepidolite, paragonite, and artificial or synthetic mica.

Silicas useful herein can include, hydrated silica, mineral silicates, amorphous silica, fumed silica, calcium silicate, magnesium silicate, magnesium aluminum silicate, magnesium trisilicate, and others. By fumed silica it is meant those high-surface area powdered silicas prepared by pyrogenic process and has a purity of 99.8% or greater. Fumed silicas are available in untreated form, or with a surface treatment to render the silica more hydrophobic. The surface area of the fumed silica is typically between 90 to about 380 m²/g. Other silicas useful herein include, but are not limited to mineral silicates such as phyllosilicates and tectosilicates.

Pigments useful herein include inorganic and organic pigments. The organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified color additives, referred to as the Lakes or iron oxides. A pulverulent coloring agent, such as carbon black, chromium or iron oxides, ultramarines, manganese pyrophosphate, iron blue, and titanium dioxide, pearlescent agents, generally used as a mixture with colored pigments, or some organic dyes, generally used as a mixture with colored pigments and commonly used in the cosmetics industry, can be added to the composition. Pulverulent inorganic or organic fillers can also be added. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked. Mention may be made in particular of talc, mica, silica, kaolin, nylon powders (in particular ORGASOL), polyethylene powders, Teflon, starch, boron nitride, copolymer microspheres such as EXPANCEL (Nobel Industrie), POLYTRAP, and silicone resin powder or microbeads (TOSPEARL from Toshiba).

Natural skin care powders useful herein include, but are not limited to almond meal, apricot seed powder, barley flour, corn cob meal, corn cob powder, corn flour, corn meal, jojoba seed powder, oat bran, oat flour, oatmeal, peach pit powder, pecan shell powder, rice bran, rye flour, soy flour, total soy powder, walnut shell powder, wheat bran, wheat flour, wheat starch and others.

The cosmetically acceptable powder may contain other organic and inorganic powders that are useful in skin care applications.

The amount of cosmetically acceptable powder used in the liquid-to-powder composition is typically from 50 wt % to 70 wt % based on the wt of liquid-to-powder composition, alternatively from 55 to 65 wt %, alternatively from 60 to 63 wt %. Although it may be possible to use amount higher than 70 wt %, typically the composition loses its esthetic appeal as it becomes too hard or cakey.

Component (B) in the liquid-to-powder formulation is a crosslinked silicone elastomer. Suitable crosslinked silicone elastomers useful herein are non-emulsifying crosslinked organopolysilxoanes. By non-emulsifying it is meant crosslinked silicone elastomers that do not contain polyoxyalkylene units. Non-emulsifying elastomers are typically formed via reacting an organohydrogenpolysilxoanes with a divinyl compound such as an alpha, omega diene. The crosslinked silicone elastomers may be further processed by subjecting them to high pressure to result in a elastomer with an average particle size in the range of 0.2 to 10 microns, alternatively 0.5 to 5 microns. Non-emulsifying elastomers can also be known as dimethicone crosspolymers, vinyldimethicone crosspolymers and others. Typically the crosslinked silicone elastomer will have an average molecular weight of 2,000 to in excess of 1,000,000, alternatively 10,000 to 20 million. Suitable crosslinked silicone elastomers are commercially available and methods for making the are know in the art. U.S. Pat. No. 5,654,362 is hereby incorporated by reference for its teaching on how to make the elastomers.

The crosslinked silicone elastomers are typically available delivered as 20 to 45 wt % in a silicone fluid carrier such as cyclomethicone or dimethicone. The amount of crosslinked silicone elastomer is typically from 5 to 15 wt % (solids) based on the liquid-to-powder composition. If an insufficient amount of crosslinked silicone elastomer is used then the cosmetically acceptable powder will precipitate out of the composition.

Component (C) is a volatile fluid. By “volatile” it is meant those materials that have a measurable pressure at ambient conditions. Volatile fluids useful herein include volatile silicone fluids, volatile organic fluids and mixtures thereof. Volatile silicone fluids may be cyclic, linear or mixtures thereof. Cyclic silicone fluids include polydimethylsiloxanes containing from 3 to 9 silicon atoms, alternatively 4 to 5 silicon atoms. Linear silicone fluids include polydimethylsiloxanes containing from 3 to 9 silicon atoms. The linear volatile silicones typically have viscosities of less than 5 Cs at 25° C., while the cyclic silicone fluids have viscosities of less than 10 Cs at 25° C., alternatively 0.1 to 8 Cs. Volatile silicone fluids are commercially available.

Volatile silicone fluids useful herein may be exemplified by, but not limited to hexamethyldisiloxane, octamethyltrisiloxane, decamethyltetrasiloxane, dodecamethylpentasiloxane, tetradecamethylhexasiloxane, hexadecamethylheptasiloxane, octamethylcyclotetrasiloxane, decamethylcyclopentasiloxane, dodecamethylcyclohexasiloxane, heptamethyl-3-{(trimethylsilyl)oxy}trisiloxane, hexamethyl-3,3,bis{(trimethylsilyl)oxy}trisiloxane, pentamethyl{(trimethylsilyl)oxy}cyclotrisiloxane and mixtures thereof.

The organic solvent can be an ester, an alcohol such as methanol, ethanol, isopropanol, butanol, or n-propanol, a ketone such as acetone, methylethyl ketone, or methyl isobutyl ketone; an aromatic hydrocarbon such as benzene, toluene, or xylene; an aliphatic hydrocarbon such as heptane, hexane, or octane; a glycol ether such as propylene glycol methyl ether, dipropylene glycol methyl ether, propylene glycol n-butyl ether, propylene glycol n-propyl ether, or ethylene glycol n-butyl ether, an acetate, such as ethyl acetate or butyl acetate, a halogenated hydrocarbon such as dichloromethane, 1,1,1-trichloroethane or methylene chloride, chloroform, dimethyl sulfoxide, dimethyl formamide, acetonitrile, tetrahydrofuran, or an aliphatic hydrocarbon such as white spirits, mineral spirits, isododecane heptane, hexane or naphtha.

The volatile fluid is typically present in the liquid-to-powder composition in an amount of 10 to 35 wt % based on the weight of the liquid-to-powder composition, alternatively 15 to 25 wt %. If a volatile silicone fluid is used as the carrier for the crosslinked silicone elastomer (B) then it may not be necessary to add additional amounts of volatile silicone fluid.

Component (D) is a nonvolatile silicone fluid. Nonvolatile silicone fluids useful herein can be polyalkyl siloxanes, polyalkylaryl siloxanes or polyether siloxane copolymers. The nonvolatile silicone fluids have a viscosity of typically 10 to 100,000 Cs at 25° C. The amount of nonvolatile silicone fluid is typically from 1 wt % to 10% based on the weight of the liquid-to-powder composition. Typically, the higher the viscosity of the nonvolatile silicone fluid, the less of the fluid that will be needed in the composition.

Nonvolatile silicone fluids useful herein may be exemplified by, but not limited to, dimethylpolysiloxanes, methylethylpolysiloxanes, polydiphenylsiloxanes, and mixtures thereof. Nonvolatile silicone fluids useful herein may also include those nonvolatile silicone fluids that contain a functional group, so long as the functional group is not reactive with any other component used in the liquid-to-powder composition. Functional groups that may be incorporated onto the nonvolatile silicone fluid include, but are not limited to, acrylamide groups, acrylate groups, amide groups, amide groups, amino groups, carbinol groups, carboxy groups, chloroalkyl groups, epoxy groups, glycol groups, ketal groups, mercapto groups, methyl ester groups, perfluoro groups, polyisobutylene groups, silanol groups, vinyl groups and mixtures thereof.

Additional components may be added to the liquid-to-powder composition so long as they do not cause the powder to separate out of the composition. Examples of useful additives include antibacterial agents, pigments, fragrances, anti-caking agents, spherical powders that can aid in enhancing the feel of the compositions, anti-whitening agents, light scattering agents, topically active agents, pharmaceutical actives, moisturizing conditions, exfoliants, sunscreens, deodorants, astringents, absorbing agents, vitamins, zinc oxide and mixtures thereof.

By modifying the amount and type of volatile and nonvolatile silicone fluids, the resulting appearance of the liquid powder on the skin upon drying can be varied to leave a powder residue on the skin, or give the appearance of no powdery residue at all (dry powdery feel without the presence of the residue).

The liquid-to-powder compositions are typically prepared by preparing a mixture of the silicone components (B), (C) and (D) and then slowly adding the cosmetically acceptable powder (A) to the silicone mixture. By this method, clumping of the powder can be prevented. The optional components may be blended with the silicone components, the cosmetically acceptable powder or added after the blending of the silicone and powder. The preparation of the liquid-to-powder composition can typically take place at room temperature and pressure.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %.

### Example 1

### Baby Powder Putty (Series I)

Liquid to powder compositions were prepared by dispersing (D) into a mixture of (B) and (C) in the amounts (wt %) given in Table 1 and mixing until uniform. The baby powder (A) was slowly added to the fluid mixture in increments, with stirring. After about half of the required baby powder was added (which equates to 30-35% of the formulation), the product had a smooth/fluffy texture. However, as the level approached the 60%, the putty consistency appeared.
40 grams of baby powder=very smooth lotion consistency,
50 grams of baby powder=very smooth, thicker, but still creamy

60 grams of baby powder=viscous, but very smooth putty consistency.

TABLE 1
Formulations for Example 1

| | | | | (D) |
|---|---|---|---|---|
| | | | | Dimethicone |
| | (A) | (B) | (C) | and |
| | Baby Powder | Dimethicone | Dimethicone | Dimethicone |
| Example | (Corn Starch) | and trisiloxane | (350 cSt) | crosspolymer |
| 1-1 | 63.2 | 25.1 | 1 | 10.7 |
| 1-2 | 63 | 25 | 5 | 7 |

### Example 2

### Baby Powder Putty (Series II)

These formulations resulted in smoother esthetics and anti-whitening benefits.

Liquid to powder compositions were prepared by dispersing (D) into a mixture of (B) (C), and (E) in the amounts (wt %) given in Table 2 and mixing until uniform. The baby powder (A) was slowly added to the fluid mixture in increments, with stirring. In Example 2-3, the phenyltrimethicone reduced the whitening of the composition after application to the skin.

TABLE 2
Formulations for Example 2

| | (A) | | | | |
|---|---|---|---|---|---|
| | Baby | (B) | | (D) | |
| | Powder | Dimethicone | (C) | Dimethicone and | |
| | (Corn | and | Dimethicone | Dimethicone | (E) |
| Example | Starch) | trisiloxane | (350 cSt) | crosspolymer | Phenyltrimethicone |
| 2-1 | 63 | 25 | 5 | 7 | 0 |
| 2-2 | 60.5 | 28.15 | 2 | 9.35 | 0 |
| 2-3 | 60.5 | 27.5 | 2 | 9 | 1 |

That which is claimed is:

1. A liquid-to-powder composition comprising
(A) at least one cosmetically acceptable powder
(B) at least one crosslinked silicone elastomer;
(C) at least one volatile fluid and;
(D) at least one nonvolatile silicone fluid.

2. The composition as claimed in claim 1 wherein the cosmetically acceptable powder is selected from cornstarch, talc, silica, inorganic pigments, mica, sugars, natural skin treatment powders and mixtures thereof.

3. The composition as claimed in claim 1 wherein the cosmetically acceptable powder is talc.

4. The composition as claimed in claim 1 wherein the cosmetically acceptable powder is cornstarch.

5. The composition as claimed in claim 1 wherein the cosmetically acceptable powder has a particle size of 6 to 600 microns.

6. The composition as claimed in claim 1 wherein the cosmetically acceptable powder is present in an amount of 50 wt % to 70 wt % based on the total weight of (A), (B), (C) and (D).

7. The composition as claimed in claim 1 wherein the crosslinked silicone elastomer is a non-emulsifying crosslinked organopolysiloxane.

8. The composition as claimed in claim 1 wherein the crosslinked silicone elastomer is a dimethicone crosspolymer.

9. The composition as claimed in claim 1 wherein the crosslinked silicone elastomer has an average molecular weight of 2,000 or greater.

10. The composition as claimed in claim 9 wherein the crosslinked silicone elastomer has an average molecular weight of 10,000 to 20 million.

11. The composition as claimed in claim 1 wherein the crosslinked silicone elastomer is present in an amount of 5 to 15 wt % based on the total weight of (A), (B), (C) and (D).

12. The composition as claimed in claim 1 wherein the volatile fluid is selected from cyclic silicone fluids, linear silicone fluids and mixtures thereof.

13. The composition as claimed in claim 12 wherein the volatile fluid is a cyclic silicone fluid.

14. The composition as claimed in claim 13 wherein the cyclic silicone fluid is selected from octamethylcyclotetrasiloxane, decamethylcyclopentasiloxane, dodecamethylcyclohexasiloxane and pentamethyl{(trimethylsilyl)oxy}cyclotrisiloxane.

15. The composition as claimed in claim 12 wherein the volatile fluid is a linear silicone fluid.

16. The composition as claimed in claim 15 wherein the volatile fluid is selected from hexamethyldisiloxane, octamethyltrisiloxane, decamethyltetrasiloxane, dodecamethylpentasiloxane, tetradecamethylhexasiloxane, hexadecamethylheptasiloxane, and heptamethyl-3-{(trimethylsilyl)oxy}trisiloxane, hexamethyl-3,3,bis{(trimethylsilyl)oxy}trisiloxane.
